(19) United States
(12) Patent Application Publication
(10) Pub. No.: US 2025/0295119 A1
(43) Pub. Date: Sep. 25, 2025

(54) SPHERICAL NANOPARTICLES DERIVED FROM TMGMV IMPROVE SOIL TRANSPORT OF SMALL, HYDROPHOBIC AGROCHEMICALS

(71) Applicant: THE REGENTS OF THE UNIVERSITY OF CALIFORNIA, Oakland, CA (US)

(72) Inventors: Nicole Steinmetz, San Diego, CA (US); Adam Caparco, San Diego, CA (US)

(21) Appl. No.: 18/862,453

(22) PCT Filed: May 1, 2023
(86) PCT No.: PCT/US2023/020592

§ 371 Date: Nov. 1, 2024

Related U.S. Application Data
(60) Provisional application No. 63/337,488, filed on May 2, 2022.

(51) Int. Cl. A01N 63/40; A01N 43/22; A01P 5/00; C12N 7/00

(52) U.S. Cl. CPC A01N 63/40; A01N 43/22; A01P 5/00; C12N 7/00; C12N 2770/00023; C12N 2770/00031

(57) ABSTRACT

Applicants describe herein TMGMV-derived spherical nanoparticles (SNPs) that used entrap small molecule cargo for improved loading efficiency while minimizing upstream processing steps.

### CROSS-REFERENCE TO RELATED APPLICATION

This application claims priority under 35 U.S.C. § 119 (e) to U.S. Provisional Application No. 63/337,488, filed May 2, 2022, the contents of which are incorporated herein by reference in their entireties.

### STATEMENT OF GOVERNMENT SUPPORT

This invention was made with government support under NIFA-2020-67021-31255 awarded by the National Institute of Food and Agriculture, and DMR2011924 awarded by the National Science Foundation. The government has certain rights in the invention.

### INCORPORATION BY REFERENCE

All publications, patents, and patent applications mentioned in this specification and attached Appendices are herein incorporated by reference to the same extent as if each individual publication, patent, patent application or appendix, was specifically and individually indicated to be incorporated by reference.

### BACKGROUND

Damages from plant parasitic nematodes amount to hundreds of billions of dollars in crop losses, presenting both an economic risk and a risk to the global food supply. To improve the transport behavior and sustainable use of compounds for nematode treatment, more advanced delivery vehicles to help retain hydrophobic cargo in the soil and prevent leaching into the groundwater are needed. This disclosure satisfies this need and provides related advantages as well.

### SUMMARY OF THE DISCLOSURE

One avenue to address this unmet need is to use virus-like particles and viral nanoparticles as versatile platforms for drug delivery and small-molecule loading. Due to their symmetry, reactive amino acids on their surface, and high stability compared to other proteinaceous delivery vehicles, they have demonstrated many successful applications as biocompatible and immunogenic platforms for cancer therapy, diagnostic imaging agents, and hydrogels and polymeric materials for slow release.

Recently, rod-shaped plant virus nanoparticles derived from Tobacco mild green mosaic virus (TMGMV) have delivered electrostatically and covalently bound small molecules in soil, demonstrating higher mobility than their icosahedral counterparts and other nanoparticle delivery systems. As TMGMV is currently approved by the US Environmental Protection Agency as an herbicide and it is commercially available, this platform has potential to be translated into a drug delivery platform for soil and agricultural applications. Electrostatically bound crystal violent (TMGMV-CV) effectively treated C. elegans, a model organism for nematodes, in a motility assay, showing the bound cargo retained its potency. However, improving the covalent loading efficiency of hydrophobic cargo on the virus exterior by bioconjugation is challenging due to solubility limits and the necessary molar equivalents required to drive the bioconjugation reactions. Therefore, establishing a low-cost method for loading of hydrophobic cargo to TMGMV is needed to circumvent issues of solubility and reaction efficiency.

Applicants describe herein TMGMV-derived spherical nanoparticles (SNPs) that used entrap small molecule cargo for improved loading efficiency while minimizing upstream processing steps. First, the TMGV-SNPs are prepared by thermal reshaping at several concentrations to create a range of nanoparticle diameters to work with, and the resulting particles are analyzed using electron microscopy to characterize the batch. To compare the release behavior of entrapped versus covalently attached molecules, SNPs are prepared using two methods: 1) with simple entrapment of Cyanine 5 during the thermal reshaping and 2) TMGMV is covalently linked to Cyanine 5 before thermal reshaping. The release behavior is characterized using dialysis and the soil mobility profile is characterized using SDS-PAGE and absorbance measurements. Uptake of these SNPs into C. elegans, a model nematode, is determined by fluorescent microscopy measurements. As a final measure, nematicides are loaded onto the SNPs using method 1, and the soil mobility and release profile is determined by HPLC. These results show SNPs can be deployed as a simple platform for encapsulation of otherwise insoluble compounds for drug delivery in the soil, demonstrating desirable drug release and soil mobility characteristics.

Thus, this disclosure provides a virus-like particle (VLP or “SNP”) derived from Tobacco mild green mosaic virus (TMGMV) or a derivative thereof optionally conjugated to an agent. In one aspect, the agent or derivative thereof is entrapped in the VLP or covalently attached to the VLP. In another aspect, the diameter of VLP nanoparticle can range from about 1 nm to about 2 μm. In one embodiment, the agent is selected from the list of agents or pesticides provided in Table 1, or an avermectin or a derivative thereof. In another aspect, the avermectin is selected from the group of ivermectin, abamectin, doramectin, eprinomectin, moxidectin, or selamectin.

In one aspect, the agent is selected from the list of agents or pesticides provided in Table 1, or an avermectin, or derivative thereof. In another aspect, the avermectin is selected from the group of ivermectin, abamectin, doramectin, eprinomectin, moxidectin, or selamectin. In a further aspect, the agent is ivermectin.

In a further aspect, the agent or pesticide is loaded at a concentration of about 5 mg mL⁻¹ to about 10 mg mL⁻¹ or alternatively, wherein the agent is loaded at a concentration of from about 0.5 mg mL⁻¹ to about 0.2 mg mL⁻¹.

Also provided is a plurality of VLPs that may be the same or different from each other.

Provided herein is a method of treating an agricultural environment such as example soil, feed or plants (leaf, stalk or roots), comprising, or consisting essentially of, or yet further consisting of delivering or contacting the environment a VLP formulation or a composition as described herein. They also are useful to deliver pesticides and other insoluble compounds to inhibit pathogenic infestations of plants, roots, and soil. In one aspect the method is performed with a plurality of VLPs as described herein, wherein the VLPs are the same or different from each other and/or the agents are the same or different from each other and/or the TMGMV or a derivative thereof are the same or different.

As is apparent to the skilled artisan, the agent or pesticide is selected for the treatment of an infection or pest, see for example those identified in Table 2.

### BRIEF DESCRIPTION OF THE DRAWINGS

FIGS. 1A-1D: A schematic representation of thermally induced rod-to-SNP transition of TMGMV (FIG. 1A). A representative phase space of TMGMV-to-SNP transition at 96° C. (FIG. 1B, left) and at 30 s of heating time (FIG. 1B, right), with dark grey indicating no formation, lightest grey indicating partial transformation, and medium grey indicating reliable SNP formation. A phase space of TMGMV/SNP morphology at 5 mg mL-1 initial TMGMV concentration after heating for 10-60 seconds and temperature ranges from 60-96° C. (FIG. 1C). A zoomed in view to demonstrate nanoparticle features of the boxes in the phase space (FIG. 1D).

FIGS. 2A-2F: Soil mobility measurements of SNPs in 10 cm of soil. A schematic of SNP delivery and fraction collection from a soil column (particles not to scale) (FIG. 2A). The calculated relative band intensity for each collected 2 mL fraction from the soil columns after loading SNPs (FIG. 2B). The gels for band intensity analysis and soil mobility of SNPs at (FIG. 2C) 0.1 mg mL-1, (FIG. 2D) 1 mg mL-1, (FIG. 2E) 5 mg mL-1, and (FIG. 2F) 10 mg mL-1. The numbers on the gel images represent the elution volume of the soil column in mL. The marker on each gel is SeeBlue2 Plus (Invitrogen) in 1×MOPS, and each of the protein fractions are around 17.5 kDa.

FIGS. 3A-3G: Encapsulation of small molecules using TMGMV SNPs. A schematic of small molecule encapsulation in SNPs, where the blue shapes represent small molecules (not to scale) (FIG. 3A). SEM micrographs of SNPs containing Cy5 (with blue pellet of SNPs inset) (FIG. 3B) and ivermectin (FIG. 3C). The encapsulation efficiency of Cy5 (1 mg mL-1) in TMGMV SNPs at several concentrations of TMGMV (FIG. 3D). Competitive ELISA results for ivermectin loading in SNPs (samples prepared to be 50 μM) (FIG. 3E). The [TMGMV], SNP diameter, and calculated number of small molecules per coat protein of TMGMV (2100 per virus particle) for Cy5 and ivermectin (FIG. 3F). Overlapping soil mobility of Cy5 signal and SNP signal as a function of elution volume in a 10 cm soil column (FIG. 3G).

FIGS. 4A-4F: Assessment of ivermectin SNPs on C. elegans paralysis. A schematic of the pluronic F127 gel burrowing assay with an attractant of E. coli on the surface (FIG. 4A). Image analysis workflow to identify the area covered by C. elegans on the surface of the pluronic F127 gel at 10× magnification; bright field (FIG. 4B), edge detected (FIG. 4C), and binary (FIG. 4D). The calculated surface velocity (FIG. 4E) and number of nematodes (FIG. 4F) for C. elegans treated with SNPs at different concentrations of ivermectin in four preparations. For FIGS. 4E and 4F, from left to right, standards, 100-200 nm, 200-500 nm, 500-1 μm and 1 μm-2 μm.

FIGS. 5A-5B: Additional SEM images of TMGMV transformations under different heating temperatures (columns) and heating times (rows). The samples shown had initial TMGMV concentrations of 1.0 mg mL-1 (FIG. 5A) and 0.1 mg mL-1 (FIG. 5B). The images are around 5000× magnification.

FIG. 6: SEM images of the effect of heating time on 10 mg mL-1 TMGMV samples during SNP formation.

FIGS. 7A-7B: Analysis of the SNP formation on the sequence and integrity of TMGMV coat protein. SDS-PAGE showing the expected molecular weight (band 1) is a majority product for each condition, although SNP formation does lead to some degradation (band 2) and dimerization (top band) (FIG. 7A). A sequence coverage map after LC/MS/MS and proteomic analysis (FIG. 7B).

FIGS. 8A-8B: Soil mobility measurements of SNPs in 10 cm of soil. The gels for band intensity analysis and soil mobility of TMGMV (FIG. 8A). The numbers on the gel image represent the elution volume of the soil column in mL. The marker on each gel is SeeBlue2 Plus (Invitrogen) in 1×MOPS, and each of the protein fractions are around 17.5 kDa. The calculated relative band intensity for each collected 2 mL fraction from the soil columns after loading TMGMV (FIG. 8B).

FIGS. 9A-9E: SEM images of ivermectin and ivermectin loaded SNP samples. The images show ivermectin after heating (FIG. 9A), ivermectin after heating with TMGMV (FIG. 9B), ivermectin and TMGMV heated with 25% MeCN (FIG. 9C), and the yield of SNPs from these conditions at 2 TMGMV concentrations (FIG. 9D, FIG. 9E). The magnification was 5000× (a-c) and 1000× (FIGS. 9D-9E).

FIG. 10: Standard curve for the competitive ELISA for ivermectin detection. Plotted is the absorbance at 450 nm vs. ivermectin concentration in μM after 30 minutes of reaction. Each condition was conducted in triplicate, averages and standard deviation are shown (for some data points errors were smaller than the data points).

FIGS. 11A-11B: CD spectra of TMGMV and SNP samples. Legends are located below the graphs. Far UV spectra (FIG. 11A) and Near UV spectra (FIG. 11B).

FIG. 12: Soil mobility of IVN-SNPs in a 20 cm soil column.

FIGS. 13A-13D: Example micrographs of C. elegans on Pluronic gel surface. The tracks of their movement are the undulating lines. The animals are denoted by the red arrow. Each frame (FIGS. 13A-13D) represents a different area of the same treatment group.

FIG. 14: Example image of a Pluronic F127 gel surface before breakthrough of C. elegans.

FIGS. 15A-15E: Example area scans of C. elegans burrowing assays after treatment the animals with ivermectin solution (FIGS. 15A-15D) and a TMGMV only control (FIG. 15E). 0 μM (FIG. 15A), 0.1 μM (FIG. 15B), 1.0 μM (FIG. 15C), 10 μM (FIG. 15D). Each experiment was conducted in triplicate.

FIGS. 16A-16E: Example area scans of C. elegans burrowing assays after treatment the animals with SNP 0.1 samples loaded with ivermectin. 0 μM (FIG. 16A), 0.1 μM (FIG. 16B), 1.0 μM (FIG. 16C), 10 μM (FIG. 16D), 10 μM after soil column (FIG. 16E). Each experiment was conducted in triplicate.

FIGS. 17A-17E: Example area scans of C. elegans burrowing assays after treatment the animals with SNP 1.0 samples loaded with ivermectin. 0 μM (FIG. 17A), 0.1 μM (FIG. 17B), 1.0 μM (FIG. 17C), 10 μM (FIG. 17D), 10 μM after soil column (FIG. 17E). Each experiment was conducted in triplicate.

FIGS. 18A-18E: Example area scans of C. elegans burrowing assays after treatment the animals with SNP 5.0 samples loaded with ivermectin. 0 μM (FIG. 18A), 0.1 μM (FIG. 18B), 1.0 μM (FIG. 18C), 10 μM (FIG. 18D), 10 μM after soil column (FIG. 18E). Each experiment was conducted in triplicate.

FIGS. 19A-19E: Example area scans of C. elegans burrowing assays after treatment the animals with SNP 10 samples loaded with ivermectin. 0 μM (FIG. 19A), 0.1 μM (FIG. 19B), 1.0 μM (FIG. 19C), 10 μM (FIG. 19D), 10 μM after soil column (FIG. 19E). Each experiment was conducted in triplicate.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the term “comprising” is intended to mean that the methods include the recited steps or elements, but do not exclude others. “Consisting essentially of” shall mean rendering the claims open only for the inclusion of steps or elements, which do not materially affect the basic and novel characteristics of the claimed methods. “Consisting of” shall mean excluding any element or step not specified in the claim. Embodiments defined by each of these transition terms are within the scope of this disclosure.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

An “agricultural product” intends vegetation in whole or in part and includes plants, trees, roots, flowers, limbs, shoots, stems, leaves or any other part thereof.

As used herein, the terms “treating,” “treatment” and the like mean obtaining a desired agricultural effect such as an amelioration. The effect may be prophylactic in terms of completely or partially preventing an infection of a plant, vegetation, or other agricultural product by a pest or insect. In one aspect, the term “treatment” excludes prophylaxis.

The term “ameliorate” means a detectable improvement in an agricultural product or vegetation, such as a plant, tree, flower, crop, root, stem, or leaf. A detectable improvement includes a subjective or objective decrease, reduction, inhibition, suppression, limit or control in the occurrence, frequency, severity, progression, or duration of an infection or presence of a pest or microorganism, such as for example C. elegans.

As used herein, the term “agent” intends an active agent that treats or ameliorate the presence of or infection of an agricultural product, e.g., a pesticide or an insecticide, e.g. avermectin such as ivermectin.

Common insecticides are described in Table 1, below:

TABLE 1
Insecticide Types and Their Modes of Action

| Insecticide Type | Mode of Action |
|---|---|
| Organochlorine | Most act on neurons by causing a sodium/potassium imbalance |
| | preventing normal transmission of nerve impulses. Some act on the |
| | GABA (γ-aminobutyric acid) receptor preventing chloride ions from |
| | entering the neurons causing a hyperexcitable state characterized by |
| | tremors and convulsions. Usually broad-spectrum insecticides that have |
| | been taken out of use. |
| Organophosphate | Cause acetylcholinesterase (AChE) inhibition and accumulation of |
| | acetylcholine at neuromuscular junctions causing rapid twitching of |
| | voluntary muscles and eventually paralysis. A broad-range insecticide, |
| | generally the most toxic of all pesticides to vertebrates. |
| Organosulfur | Exhibit ovicidal activity (i.e., they kill the egg stage). Used only against |
| | mites with very low toxicity to other organisms. |
| Carbamates | Cause acetylcholinesterase (AChE) inhibition leading to central |
| | nervous system effects (i.e., rapid twitching of voluntary muscles and |
| | eventually paralysis). Has very broad spectrum toxicity and is highly |
| | toxic to fish. |
| Formamidines | Inhibit the enzyme monoamine oxidase that degrades neurotransmitters |
| | causing an accumulation of these compounds; affected insects become |
| | quiescent and die. Used in the control of OP and carbamate-resistant |
| | pests. |
| Dinitrophenols | Act by uncoupling or inhibiting oxidative phosphorylation preventing |
| | the formation of adenosine triphosphate (ATP). All types have been |
| | withdrawn from use. |
| Organotins | Inhibit phosphorylation at the site of dinitrophenol uncoupling, |
| | preventing the formation of ATP. Used extensively against mites on |
| | fruit trees and formerly used as an antifouling agent and molluscacide; |
| | very toxic to aquatic life. |
| Pyrethroids | Acts by keeping open the sodium channels in neuronal membranes |
| | affecting both the peripheral and central nervous systems causing a |
| | hyper-excitable state. Symptoms include tremors, incoordination, |
| | hyperactivity and paralysis. Effective against most agricultural insect |
| | pests; extremely toxic to fish. |
| Nicotinoids | Act on the central nervous system causing irreversible blockage of the |
| | postsynaptic nicotinergic acetylcholine receptors. Used in the control of |
| | sucking insects, soil insects, whiteflies, termites, turf insects and the |
| | Colorado potato beetle. Generally have low toxicity to mammals, birds |
| | and fish. |
| Spinosyns | Acts by disrupting binding of acetylcholine in nicotinic acetylcholine |
| | receptors at the postsynaptic cell. Effective against caterpillars, |
| | lepidopteran larvae, leaf miners, thrips and termites. Regarded for its |
| | high level of specificity. |
| Pyrazoles | Inhibits mitochondrial electron transport at the NADH-CoQ reductase |
| | site leading to disruption of ATP formation. Effective against psylla, |
| | aphids, whitefly and thrips. Results of testing on one type (fipronil) |
| | indicate no effects on the clams, oysters or fish, with marginal effects |
| | on shrimp. |
| Pyridazinones | Interrupt mitochondrial electron transport at Site 1; mainly used as a |
| | miticide; display toxicity to aquatic arthropods and fish. |
| Quinazolines | Acts on the larval stages of most insect by inhibiting or blocking the |
| | synthesis of chitin in the exoskeleton. Developing larvae exhibit |
| | rupture of the malformed cuticle or death by starvation; not registered |
| | in U.S. |
| Botanicals | Depending upon the type can have various effects: |
| | Pyrethrum - affects both the central and peripheral nervous systems, |
| | stimulating nerve cells to produce repetitive discharges and eventually |
| | eading to paralysis. Commonly used to control lice. |
| | Nicotine - mimics acetylcholine (Ach) in the central nervous system |
| | ganglia, causing twitching, convulsions and death. Used most to |
| | control aphids and caterpillars. |
| | Rotenone - acts as a respiratory enzyme inhibitor. Used as a piscicide |
| | that kills all fish at doses non-toxic to fish food organisms. |
| | Limonene - affects the sensory nerves of the peripheral nervous |
| | system. Used to control fleas, lice, mites and ticks, while remaining |
| | virtually non-toxic to warm-blooded animals and only slightly toxic to |
| | fish. |
| | Neem - reduces feeding and disrupts molting by inhibiting |
| | biosynthesis or metabolism of ecdysone, the juvenile molting hormone. |
| | Commonly used against moth and butterfly larvae. |
| Synergists/Activators | Inhibit cytochrome P-450 dependent polysubstrate monooxygenases |
| | (PSMOs) preventing the degradation of toxicants, enhancing the |
| | activity of insecticides when used in concert; synergists and activators |
| | are not in themselves considered toxic or insecticidal. |
| Antibiotics | Act by blocking the neurotransmitter GABA at the neuromuscular |
| | junction; feeding and egg laying stop shortly after exposure while death |
| | may take several days. Most promising use of these materials is the |
| | control of spider mites, leafminers and other difficult to control |
| | greenhouse pests. |
| Fumigants | Act as narcotics that lodge in lipid-containing tissues inducing narcosis, |
| | sleep or unconsciousness; pest affected depends on particular |
| | compound. |
| Inorganics | Mode of |
| | action is dependent upon type of inorganic: may uncouple |
| | oxidative phosphorylation (arsenicals), inhibit enzymes involved in |
| | energy production, or act as desiccants. Pest group depends on |
| | compound (e.g., sulfur for mites, boric acid for cockroaches). |
| Biorational | Grouped as biochemicals (hormones, enzymes, pheromones natural |
| | agents such as growth regulators) or microbials (viruses, bacteria, |
| | fungi, protozoa and nematodes). Act as either attractants, growth |
| | regulators or endotoxins; known for very low toxicity to non-target |
| | species. |
| Benzoylureas | Act as insect growth regulators by interfering with chitin synthesis. |
| | Greatest value is in the control of caterpillars and beetle larvae but is |
| | also registered for gypsy moth and mushroom fly. Some types are |
| | known for their impacts on invertebrates (reduced emergent species) |
| | and early life stages of sunfish (reduced weight) (Boyle et al. 1996). |

Reproduced from Insecticides, https://www.epa.gov/caddis-vol2/insecticides, last accessed on Apr. 29, 2023.

As is apparent to one of skill in the art, the agent is selected to treat or ameliorate the presence of the pest or agricultural infection. Non-limiting examples of such include an avermectin. Fermectins are a group of drugs that occurs naturally as a product of fermenting Streptomyces avermitilis, an actinomycetes, isolated from the soil. Eight different structures, including ivermectin, abamectin, doramectin, eprinomectin, moxidectin, and selamectin, are known and divided into four major components (A1a, A2a, B1a and B2a) and four minor components (A1b, A2b, B1b, and B2b). Avermectins are generally used as a pesticide for the treatment of pests and parasitic worms as a result of their anthelmintic and insecticidal properties. See, El-Saber Batiba, et al. (2020), Pharmaceuticals, August 17; 13 (8): 196. doi: 10.3390/ph13080196. PMID: 32824399; PMCID: PMC7464486. Non-limiting examples of crops and common insecticides to treat crops or vegetation are provided in Table 2.

TABLE 2
Examples of Crops and Common Insecticides Used

| Crop | Insecticides |
|---|---|
| Corn, | Permethrin (pyrethroid), Esfenvalerate (pyrethroid), Bacillus thuringiensis (BT- |
| sweet | Biologicals), Diazinon (organophosphate), Methomyl (carbamate), Malathion |
| | (organophosphate), pyrethrin (botanical), Carbaryl (N-methyl carbamate), |
| | Endosulfan (organochlorine) |
| Alfalfa | Beta-cyfluthrin (pyrethyroid), Carbaryl (carbamate), Chlorpyrifos |
| | (organophosphate), Cyfluthrin (pyrethroid), Dimethoate (organophosphate), |
| | Gama-cyhalothrin (pyrethroid), Idoxacard (carboxylate), Methomyl (carbamate). |
| | Methyl Parathion (organophosphate), Permethrin (pyrethroid), Phosmet |
| | (organophosphate), Spinosad (fermentation product), Zeta-cypermethrin |
| | (pyrethroid) |
| Sorghum | Beta-cyfluthrin (pyrethyroid), Carbaryl (carbamate), Chlorpyrifos |
| | (organophosphate), Deltamethrin (pyrethroid), Dimethoate (organophosphate), |
| | Esfenvalerate (pyrethroid), Gama- and Lamda-cyhalothrin (pyrethroid), Malathion |
| | (organophosphate), Methidathion (organophosphate), Methomyl (cyclodine), |
| | Spinosad (fermentation product), Zeta-cypermethrin (pyrethroid) |
| Sunflower | Bacillus thuringiensis (bacterium), Beta-cyfluthrin (pyrethyroid), Carbaryl |
| | (carbamate), Chlorpyrifos (organophosphate), Deltamethrin (pyrethroid), |
| | Esfenvalerate (pyrethroid), Gama- and Lamda-cyhalothrin (pyrethroid), Methyl |
| | Parathion (organophosphate), Zeta-cypermethrin (pyrethroid) |
| Wheat | Beta-cyfluthrin (pyrethyroid), Carbaryl (carbamate), Chlorpyrifos |
| | (organophosphate), Dimethoate (organophosphate), Endosulfan (chlorinated |
| | hydrocarbon), Gama- and Lamda-cyhalothrin (pyrethroid), Malathion |
| | (organophosphate), Methidathion (organophosphate), Methomyl (cyclodine), |
| | Methyl parathion (organophosphate), Spinosad (fermentation product), Zeta- |
| | cypermethrin (pyrethroid) |
| Grapes | Sevin (carbaryl), Imidan (phosmet), Kelthane (dicofol), Guthion (azinphos |
| | methyl), Vendex (hexakis fenbutatin-oxide), Lanate (methomyl), Methoxychlor |
| | (methoxychlor), Provado (imidacloprid), Thiodan (endosulfan), Malathion, |
| | Neemix, Pyrethrins |
| Citrus | Cygon 400 (dimethoate), Cythion 57% (malathion), Diazinon AG500 |
| | (organophosphate), Dibrom 8E, Dipel 2X, Imidan 50 WP, Lannate L, Lorsban 15 |
| | G, Metasystox-R, Parathion 4E, Thiodan 3E, Zolone 3EC |
| Cotton | Acramite (bifenazate); Baythroid (cyfluthrin); Dimilin (diflubenzuron); Fulfill |
| | (pymetrozine); MSR (oxydemeton-methyl); Temik (aldicarb); Venom |
| | (dinotefuran); Zeal (etoxazole) |
| Soybeans | Asana XL (esfenvalerate); Baythroid 2 (cyfluthrin); Cruiser 5FS (thiamethoxam); |
| | Dimethoate 4E (organophosphate); Gaucho 480 (imidacloprid); Lorsban 4E |
| | (chlorpyrifos); Mustang Max (pyrethroid); Nufos 4E (chlorpyrifos); Warrior |
| | (organophosphate) |

Reproduced from Insecticides, https://www.epa.gov/caddis-vol2/insecticides, last accessed on Apr. 29, 2023.

### Virus-Like Particles (VPLs) and Compositions

As used herein, the terms “SNP” or “Virus-like particle” or “VLP” refers to a non-replicating, viral shell, derived from one or more viruses (e.g., one or more plant viruses described herein). VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. VLPs can also be engineered, e.g., comprising, or consisting essentially of, or yet further consisting of, one or more viral proteins that comprise, or consists essentially of, or yet further consists of, a modification. Methods for producing VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354:53073, 2012).

As utilized herein, a VLP is a Tobacco mild green mosaic virus (TMGMV) derived VLP that comprises, or consists essentially of, or yet further consists of, one or more viral particles, e.g., a capsid, derived from Tobacco mild green mosaic virus (TMGMV) or a derivative thereof.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a VLP, e.g., a rod-shaped plant virus nanoparticles derived from Tobacco mild green mosaic virus (TMGMV) or derivative thereof.

As used herein, the term “modification” includes, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version. These or other modifications are intended in the scope “or derivative thereof.”

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/blast/Blast.cgi.

Modified capsid polypeptides include, for example, non-conservative and conservative substitutions of the capsid amino acid sequences. In one aspect, the modified capsids are within the scope of the term “derivative thereof.”

As used herein, the term “conservative substitution” denotes the replacement of an amino acid residue by another, chemically or biologically similar residue. Biologically similar means that the substitution does not destroy a biological activity or function, e.g., assembly of a viral capsid.

Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

Provided herein is a virus-like particle (VLP) derived from Tobacco mild green mosaic virus (TMGMV) or a derivative thereof optionally conjugated to an agent. In one aspect, the agent or derivative thereof is entrapped in the VLP or covalently attached to the VLP. In another aspect, the diameter of VLP nanoparticle can range from about 1 nm to about 2 μm. In certain embodiments, the nanoparticle is less than about 2 μm, or less than about 1.5 μm, or less than about 1.25 μm or less than about 1 μm, or less than about 0.9 μm, or less than about 0.8 μm, or less than about 0.7 μm, or about less than about 0.5 μm in diameter. In other embodiments, the diameter of VLP nanoparticle is less than about 500 nm, less than about 400 nm, less than about 300 nm, less than about 200 nm, less than about 100 nm, or less than about 50 nm in diameter. In one aspect, the diameter of the VLP is from about 100 nm to about 200 nm, and ranges in between. In one aspect, the agent is selected from the list of agents or pesticides provided in Table 1, or an avermectin or a derivative thereof. In another aspect, the avermectin is selected from the group of ivermectin, abamectin, doramectin, eprinomectin, moxidectin, or selamectin.

In one aspect, the agent is selected from the list of agents or pesticides provided in Table 1, or an avermectin, or derivative thereof. In another aspect, the avermectin is selected from the group of ivermectin, abamectin, doramectin, eprinomectin, moxidectin, or selamectin. In a further aspect, agent is ivermectin. In a further aspect, the VLP is TMGMV or a derivative thereof, the agent is ivermectin and the diameter of the nanoparticle is from about 100 nm to about 200 nm, and ranges in between. In another aspect, the nanoparticle is TMGMV or a derivative thereof, the agent is an avermectin such as ivermectin and the diameter of the nanoparticle is from about 100 nm to about 200 nm, and ranges in between.

In a further aspect, the agent or pesticide is loaded at a concentration of about 5 mg mL⁻¹ to about 10 mg mL⁻¹ or alternatively, wherein the agent is loaded at a concentration of from about 0.5 mg mL⁻¹ to about 0.2 mg mL⁻¹. In one aspect, the agent is selected from the list of agents or pesticides provided in Table 1, or an avermectin, or derivative thereof loaded at a concentration of about 5 mg mL⁻¹ to about 10 mg mL⁻¹ or alternatively, wherein the agent is loaded at a concentration of from about 0.5 mg mL⁻¹ to about 0.2 mg mL⁻¹. In another aspect, the avermectin is selected from the group of ivermectin, abamectin, doramectin, eprinomectin, moxidectin, or selamectin of about 5 mg mL⁻¹ to about 10 mg mL⁻¹ or alternatively, wherein the agent is loaded at a concentration of from about 0.5 mg mL⁻¹ to about 0.2 mg mL⁻¹. In a further aspect, agent is ivermectin which is loaded at a concentration of about 5 mg mL⁻¹ to about 10 mg mL⁻¹ or alternatively, wherein the agent is loaded at a concentration of from about 0.5 mg mL⁻¹ to about 0.2 mg mL⁻¹. In a further aspect, the VLP is TMGMV or a derivative thereof, the agent is ivermectin and the diameter of the nanoparticle is from about 100 nm to about 200 nm, and ranges in between and the agent is an avermectin such as ivermectin and the diameter of the nanoparticle is from about 100 nm to about 200 nm, and ranges in between.

In some instances, an VLP described herein further comprise, or consists essentially of, or yet further consists of, a label or a tag, e.g., such as a detectable label. A detectable label can be attached to, e.g., to the surface of a VLP. In one aspect, a rod-shaped plant virus nanoparticles derived from Tobacco mild green mosaic virus (TMGMV) or derivative thereof further comprises, consists essentially thereof, or consists of the label or tag.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, ¹¹³I, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodymium, indium, or technetium. In additional embodiments, a metal oxide includes one or more of: Gd(III), Mn(II), Mn(III), Cr(II), Cr(III), Cu(II), Fe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the VLP. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetraacetic acid.

Also provided herein is the VLP as described herein further comprising, or consisting essentially of, or yet further consisting of an additional agent. Non-limiting examples of such include otherwise insoluble compounds and pesticides for drug delivery in the soil, demonstrating desirable drug release and soil mobility characteristics. These are covalently linked to the VLP and/or entrapped within the VLP. The agents also can be covalently attached to the VLP by use of a linker.

The size of the VLP nanoparticle can range from about 1 nm to about 2 μm. In certain embodiments, the nanoparticle is less than about 2 μm, or less than about 1.5 μm, or less than about 1.25 μm or less than about 1 μm, or less than about 0.9 μm, or less than about 0.8 μm, or less than about 0.7 μm, or about less than about 0.5 μm in diameter. In other embodiments, the VLP nanoparticle is less than about 500 nm, less than about 400 nm, less than about 300 nm, less than about 200 nm, less than about 100 nm, or less than about 50 nm in diameter. In further embodiments, the nanoparticle is from about 75 nm to about 300 nm, or from about 75 nm to about 275 nm, or about 75 nm to about 225 nm, or from about 100 nm to about 300 nm, or from about 100 nm to about 250 nm, or from about 100 nm to about 200 nm, and ranges in between.

Also provided is a plurality of VLPs as described herein, wherein the VLPs are the same or different from each other and/or the agents are the same or different from each other and/or the TMGMV or a derivative thereof are the same or different.

### Compositions

In another aspect, provided herein is a composition comprising, consisting essentially of, or consisting of a VLP as provided herein, and at least one carrier, suitable for its intended use, e.g. in soils or other agricultural environments. In one aspect, the VLP is rod-shaped plant virus nanoparticles derived from Tobacco mild green mosaic virus (TMGMV) or derivative thereof.

Compositions comprising, or consisting essentially of, or consisting of the VLP composition alone or in combination of other agents can be manufactured by means of conventional mixing, dissolving, granulating, dragee-making levigating, emulsifying, encapsulating, entrapping, or lyophilization processes. These can be formulated in conventional manner using one or more carriers, diluents, excipients, or auxiliaries which facilitate processing of the combinations of compounds provided herein into preparations which can be used in agriculture.

In some embodiments, the agricultural formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In one aspect, the nanoparticle is combined with a carrier, such as an organic solvent (e.g., water) or mineral clay, adjuvants such as stickers or spreaders, stabilizers, safeners, or other chemicals that improve or enhance pesticidal activity. See, for example, http://npic.orst.edu/factsheets/formulations.html, and references cited therein.

In some embodiments, the composition includes a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Agriculturally compatible carrier materials include, but are not limited to, acacia, gelatin, colloidal silicon dioxide, calcium glycerophosphate, calcium lactate, maltodextrin, glycerine, magnesium silicate, polyvinylpyrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

In some instances, the compositions further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the composition includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the compositions include, but are not limited to, sugars like trehalose, sucrose, mannitol, maltose, glucose, or salts like potassium phosphate, sodium citrate, ammonium sulfate and/or other agents such as heparin to increase the solubility and stability.

In some instances, the compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL® dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the compositions include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL®PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the compositions include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the compositions described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants are included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In a further aspect, the composition is frozen or lyophilized and can contain agents to preserve stability and activity.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the compositions may be administered or delivered to an environment, either alone or as part of a formulation, once a week, once a day, twice a day, three times a day, or four times a day, or even more frequently.

Administration of the VLPs formulation alone or in combination with the additional agent and compositions containing same can be affected by any method that enables delivery to the site of action.

Thus, the skilled artisan would appreciate, based upon the disclosure provided herein, that the dose and dosing regimen is adjusted in accordance with methods well-known in the art.

### Uses of the VLPs and Compositions Containing Same

Provided herein is a method of treating an agricultural environment such as example soil, feed or plants (leaf, stalk or roots), comprising, or consisting essentially of, or yet further consisting of delivering or contacting the environment a VLP formulation or a composition as described herein. They also are useful to deliver pesticides and other insoluble compounds to inhibit pathogenic infestations of plants, roots, and soil. In one aspect the method is performed with a plurality of VLPs as described herein, wherein the VLPs are the same or different from each other and/or the agents are the same or different from each other and/or the TMGMV or a derivative thereof are the same or different.

As is apparent to the skilled artisan, the agent or pesticide is selected for the treatment of an infection or pest, see for example those identified in Table 2.

In one aspect, the method is practiced with a VLP derived from Tobacco mild green mosaic virus (TMGMV) or a derivative thereof optionally conjugated to an agent. In a further aspect, it is conjugated to the agent. In one aspect, the agent or derivative thereof is conjugated by being entrapped in the VLP or covalently attached to the VLP. In another aspect, the method of the disclosure is practiced with a VLP having a diameter from about 1 nm to about 2 μm. In certain embodiments, the nanoparticle is less than about 2 μm, or less than about 1.5 μm, or less than about 1.25 μm or less than about 1 μm, or less than about 0.9 μm, or less than about 0.8 μm, or less than about 0.7 μm, or about less than about 0.5 μm in diameter. In other embodiments, the diameter of VLP nanoparticle in the method is less than about 500 nm, less than about 400 nm, less than about 300 nm, less than about 200 nm, less than about 100 nm, or less than about 50 nm in diameter. In one aspect, the diameter of the VLP in the method is from about 100 nm to about 200 nm, and ranges in between. In one aspect, the agent in the method is selected from the list of agents or pesticides provided in Table 1, or an avermectin or a derivative thereof. In another aspect, the avermectin is selected from the group of ivermectin, abamectin, doramectin, eprinomectin, moxidectin, or selamectin. In a further aspect, the agent is an avermectin, such as ivermectin to treat C. elegans.

In one aspect, the agent used in the methods is selected from the list of agents or pesticides provided in Table 1, or an avermectin, or derivative thereof. In another aspect, the avermectin is selected from the group of ivermectin, abamectin, doramectin, eprinomectin, moxidectin, or selamectin. In a further aspect, agent used in the methods is ivermectin. In a further aspect, the VLP used in the methods is TMGMV or a derivative thereof, the agent is ivermectin and the diameter of the nanoparticle is from about 100 nm to about 200 nm, and ranges in between. In another aspect, the nanoparticle used in the methods is TMGMV or a derivative thereof, the agent is an avermectin such as ivermectin and the diameter of the nanoparticle is from about 100 nm to about 200 nm, and ranges in between.

In a further aspect, the agent or pesticide used in the methods is loaded at a concentration of about 5 mg mL⁻¹ to about 10 mg mL⁻¹ or alternatively, wherein the agent is loaded at a concentration of from about 0.5 mg mL⁻¹ to about 0.2 mg mL⁻¹. In one aspect, the agent used in the methods is selected from the list of agents or pesticides provided in Table 1, or an avermectin, or derivative thereof loaded at a concentration of about 5 mg mL⁻¹ to about 10 mg mL⁻¹ or alternatively, wherein the agent is loaded at a concentration of from about 0.5 mg mL⁻¹ to about 0.2 mg mL⁻¹. In another aspect, the avermectin used in the methods is selected from the group of ivermectin, abamectin, doramectin, eprinomectin, moxidectin, or selamectin of about 5 mg mL⁻¹ to about 10 mg mL⁻¹ or alternatively, wherein the agent used in the methods is loaded at a concentration of from about 0.5 mg mL⁻¹ to about 0.2 mg mL⁻¹ In a further aspect, agent is ivermectin used in the methods is loaded at a concentration of about 5 mg mL⁻¹ to about 10 mg mL⁻¹ or alternatively, wherein the agent used in the methods is loaded at a concentration of from about 0.5 mg mL⁻¹ to about 0.2 mg mL⁻¹. In a further aspect, the VLP used in the methods is TMGMV or a derivative thereof, the agent is ivermectin and the diameter of the nanoparticle is from about 100 nm to about 200 nm, and ranges in between and the agent is an avermectin such as ivermectin and the diameter of the nanoparticle is from about 100 nm to about 200 nm, and ranges in between.

In some instances, an VLP used in the methods described herein further comprise, or consists essentially of, or yet further consists of, a label or a tag, e.g., such as a detectable label. A detectable label can be attached to, e.g., to the surface of a VLP. In one aspect, a rod-shaped plant virus nanoparticles derived from Tobacco mild green mosaic virus (TMGMV) or derivative thereof further comprises, consists essentially thereof, or consists of the label or tag.

### Kits

As used herein, a kit or article of manufacture described herein include a carrier, package, or container that is compartmentalized to receive one or more containers such as vials, tubes, and the like, each of the container(s) comprising, or consisting essentially of, or yet further consisting of, one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of agricultural packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXPERIMENTAL

These examples are provided for illustrative purposes only and not to limit the scope of the claims provided herein.

The extensive use of pesticides in agriculture causes these toxins to accumulate on crops, in soil, as well as in drinking and groundwater, severely endangering the ecosystem and human health. The first step toward a healthier society is to enhance food security by improving quality and yields (i.e. more effective crop treatment), while protecting the environment and agricultural ecosystems (i.e. preventing the leaching and accumulation of pesticides in the environment). Most pesticides are hydrophobic and thus do not have good soil mobility. This leads to overuse. As provided herein are VLP with enhanced properties for the delivery of pesticides. In one aspect, linkers are added to load pesticides in the VLPs, using a neat ‘plug & play’ strategy.

Alternatively, provided herein is a non-covalent encapsulation techniques to encapsulate pesticides into the nanoparticles from tobacco mild green mosaic virus (TMGMV). TMGMV is a good platform for precision farming, because it has excellent soil mobility.

TMGMV rod-shaped viruses are thermally transitioned into spherical nanoparticles (termed SNPs)-during the encapsulation process hydrophobic pesticide cargoes are encapsulated at high yield; the surprising discovery also: the thermal reshaping enables covalent or covalent-like bonding of the pesticide to the carrier, which enhances soil mobility of the cargo.

Biotin or avidin can be used as molecular linkers to load pesticides onto TMGMV-VLPs. The biomolecular linkers overcome the need for direct bioconjugation which is proven challenging for pesticides (both from a chemistry point of view and regulatory procedures).

Thus, the disclosed VLPs are used as synthetic nanocarriers for pesticide delivery. The VLPs are ideal because of their unique zwitterionic nature and modular shape which allows these protein nanoparticles to have favorable soil transport.

### Experimental Discussion

As the global population increases, strategies to protect crops are essential for meeting the forthcoming demand for food. At current levels, 9.8% of people are undernourished or starving. Financial losses in the agriculture sector due to plant parasitic nematodes amount to $125B USD and 14% of crops lost each year worldwide1. The advent of pesticide use has overcome many of the challenges of crop losses, but introduces new challenges in human health, environmental toxicity, and skepticism among the public. Compounded with a robust regulatory environment, creating next-generation nanopesticides, which can assuage and address many of the concerns around crop protection, is an essential step in creating a sustainable agricultural system.

The present disclosure provides the application of nanoparticles for soil delivery of pesticides many of which suffer from poor soil mobility and rapid washout effects, necessitating a delivery vehicle to remain effective and economically viable²⁻⁵. Pesticide delivery systems derived from polymers and nanoparticles have been developed but suffer from high cost and may not have optimal soil mobility properties^{2, 4, 6}. One promising platform for nanopesticide delivery is Tobacco mild green mosaic virus (TMGMV) nanoparticles. TMGMV has many desirable characteristics as a nanocarrier for agrochemical delivery, is commercially available, and is currently approved by the United States EPA as an herbicide^{4, 7-9}. In prior studies, covalent and electrostatic strategies of cargo loading in TMGMV were investigated; model cargo molecules were explored and toxicity toward nematodes was confirmed⁹. Importantly, Applicant demonstrated herein that TMGMV exhibits good soil mobility which is of importance because nematodes infest the root systems of crops⁴. While successful, covalent and electrostatic loading was rather inefficient; and loading of agrochemicals proved challenging even with extensive formulation chemistryl¹⁰. This prior work highlights the economics of TMVGMV-based preparations need to be improved to make a commercially viable candidate for agricultural nanotechnology. An important consideration for agrochemicals is non-covalent strategies are preferred for nanoparticle formulation, as modifications to the agrochemical initiate new regulatory approval and registration processes. Without being bound by theory, Applicant explored herein the thermal shape-switching properties that have been reported for some high aspect ratio viruses such as Tobacco mosaic virus (TMV), Alternathera mosaic virus (AltMV), and Potato virus X (PVX)¹¹⁻¹³. To encapsulate agrochemical cargo during this transition, Applicant developed the protocols for SNP formulation from TMGMV. The structural properties of the SNPs were evaluated and their loading capacity was assessed by making use of a fluorophore (Cyanine 5) and a nematicide (ivermectin). Applicant demonstrates herein that soil mobility and efficacy against Caenorhabditis elegans (C. elegans) by treatment with ivermectin in SNPs.

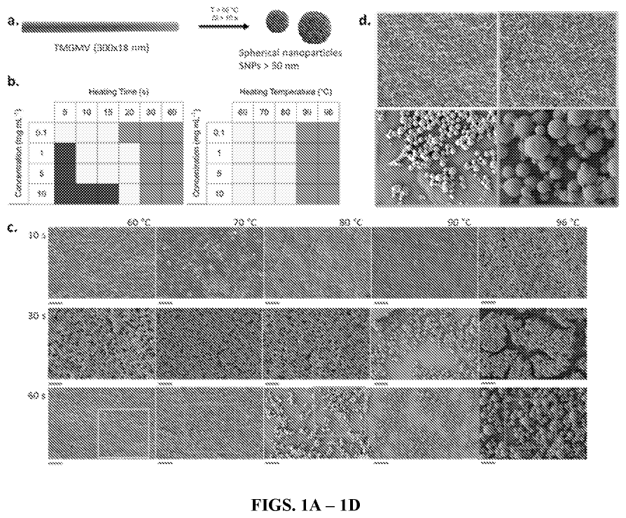

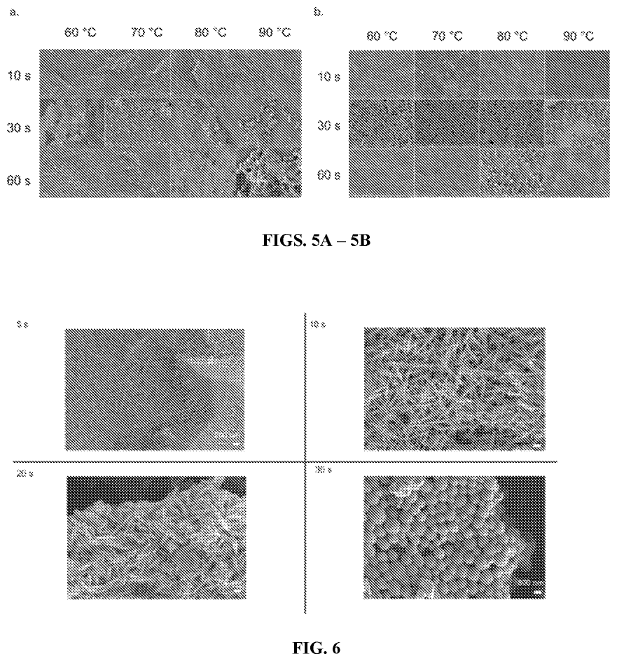

SNPs can be generated from high aspect ratio plant viruses, but data show conditions need to be optimized for each system¹³⁻¹⁶. To test whether SNPs from TMGMV could form, Applicant investigated a phase space for rod-to-SNP transition (FIG. 1). Applicant varied TMGMV concentration, heating time, and temperature and confirmed morphology by SEM. Conditions and transitions are summarized in FIG. 1B. FIG. 1C and FIG. 1D shows thermal reshaping results for TMGMV at 5 mg mL⁻¹ initial concentration (remaining conditions in FIG. 5). SNPs formed at all tested TMGMV concentrations but required a minimum temperature and heating time to completely transition. Below 80° C., SNPs did not form within 1 minute of heating, instead forming agglomerated rod bundles (FIG. 6). Above 90° C., SNPs formed after 30 seconds of heating. For temperatures and heating times below this threshold, aligned and agglomerated TMGMV bundles were observed (FIG. 1C and FIG. 6). At the threshold of 30 seconds at 96° C., SNPs were formed, and the SNP diameter was proportional to the initial TMGMV concentration: The lowest tested concentration (0.1 mg mL⁻¹) of TMGMV led to particle sizes around 100-200 nm in diameter, while the highest (5 and 10 mg mL⁻¹) tested concentrations led to particles in the 500 nm-2 μm range. The aggregation and clustering observed in the cases with smaller SNPs is a result of the drying process for SEM, but the rounded features observed in these samples are not present in any of the controls.

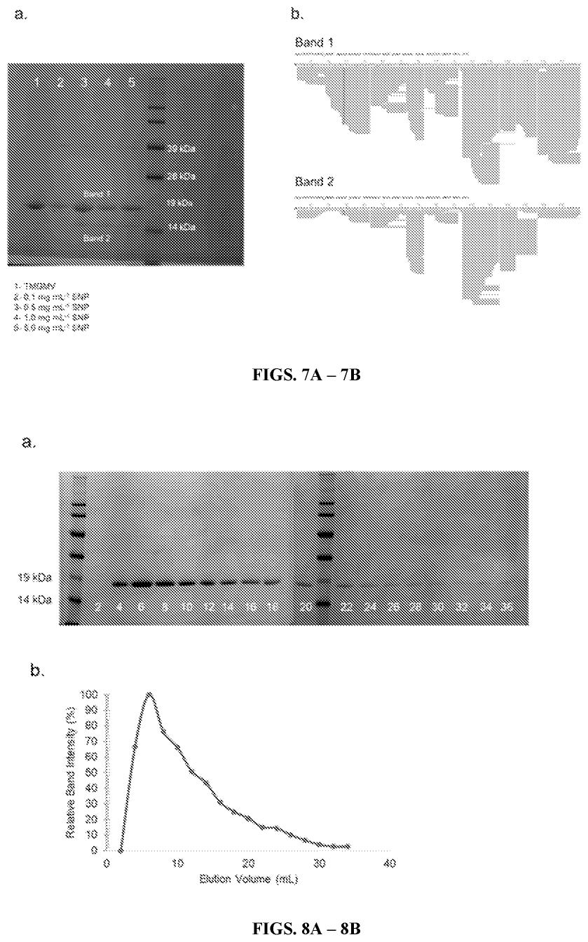

Because TMGMV SNPs were not previously reported, Applicant characterized their protein content by SDS-PAGE and LC-MS-MS. SDS-PAGE indicated differentiation between the protein content of TMGMV rods and their SNPs (FIG. 7): TMGMV consists of ˜2100 copies of an identical coat protein (CP) with a molecular weight of 17.6 kDa. SNPs have more prevalence of dimeric CP (35 kDa) even under denaturing conditions, suggesting complete disassembly was hindered. In addition, a smaller degradation byproduct (15 kDa) was observed at higher heating times. In the absence of protease or bacterial contamination, the cleaved protein sequence was unexpected. LC-MS-MS and a sequence analysis identified this band as derived from TMGMV CP. Interestingly this phenomenon has not been described for any rod-to-SNP transition¹¹⁻¹³. Aspartic acid-proline (DP) bonds in proteins can be susceptible to cleavage at temperatures close to 100° C. and in acidic conditions¹⁷⁻¹⁹ Therefore, Applicant analyzed the sequences of TMGMV and other rod-shaped viruses with reported SNP transition. TMV and TMGMV both have a DP at amino acid position D20-P21, while PVX and AltMV do not have this motif. The length of the N-terminal sequence before the putative cleavage site is 2.1 kDa, correlating with the mass shift in SDS-PAGE. This effect is reported to occur at longer heating times, on the order of minutes. Because the transition of TMGMV to SNPs takes longer than TMV, it is possible this phenomenon can occur with TMV and has not yet been observed.

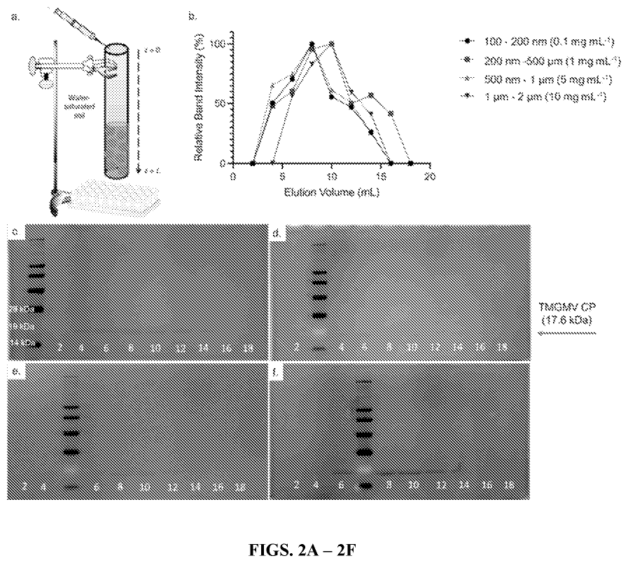

Applicant first assayed the soil mobility of SNPs vs TMGMV. It has been previously demonstrated that TMGMV has good soil mobility properties-outperforming other synthetic and biological systems and Applicant attributed this to the zwitterionic nature of the protein-based macromolecules⁴. Applicant mirrored the prior experimental set up for Applicant's soil mobility assays with SNPs⁴. In brief, a 0.3 cm diameter column was packed with Magic topsoil gardening soil (Michigan Peat) to a depth of 10 cm and pre-wetted before a bolus application of TMGMV or SNPs (0.5 mg viral nanoparticle in 1 mL DI H₂O, irrigated at 5 mL min⁻¹). Fractions were collected and analyzed for TMGMV CP by SDS-PAGE (FIG. 2 and FIG. 8). Band analysis was performed to determine the elution profile and Applicant determined that the SNPs have good soil mobility independent of their size (FIG. 2B). The size-independence may be because surface chemistry is the dominant factor when it comes to soil mobility. It is interesting to note that for 10 cm of soil depth, the SNPs display a longer elution volume (max around 8-10 mL elution volume) than the TMGMV rod-shaped particles (max around 6 mL elution volume). This is likely due to their larger length scale and change in surface chemistry of SNPs compared to rods facilitating more interactions between the particles and the soil²⁰. About 90% less overall signal intensity was observed for the 1 mg mL⁻¹ and 5 mg mL⁻¹ cases (FIG. 2D and FIG. 2E), indicating more retention of the SNPs in the soil for these conditions compared to 0.1 mg mL⁻¹ and 10 mg mL⁻¹.

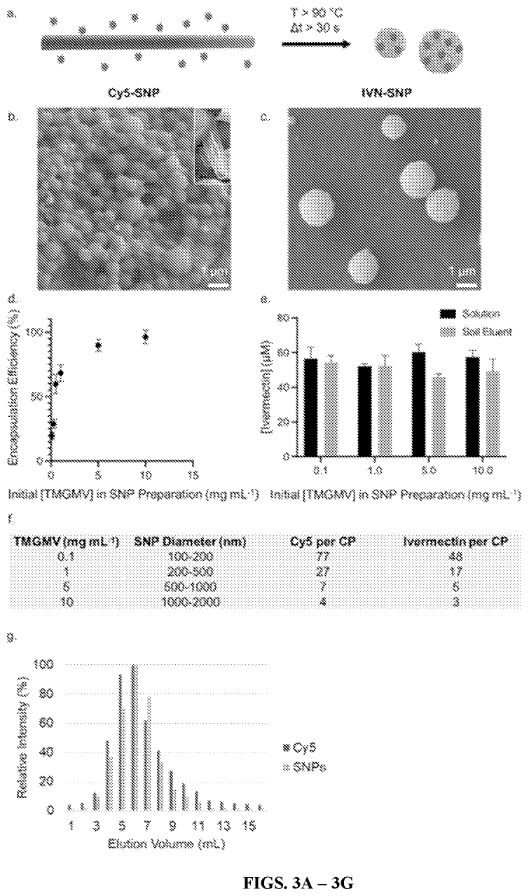

Based on the phase space, Applicant concluded the most reliable rod-to-SNP transition occurs using a 60 second heating time at 96° C. Under these conditions, SNPs formed reproducibly with uniform spheres observed on SEM. Therefore, Applicant selected the 60 sec/96° C. condition for active ingredient encapsulation as a function of protein concentration (0.1, 1.0, 5.0, and 10 mg mL⁻¹). At this protein concentration range SNPs formed ranged from 100-200 nm for the lowest protein concentration to 500 nm-2 μm for the highest protein concentration. Two small molecule cargos were selected: Cy5 was used as a fluorescent model molecule and ivermectin was selected as pesticide. Cyanine 5 (Cy5) was selected because it is a photostable fluorophore which has previously been demonstrated to remain fluorescent after heat treatments²¹. Small molecule loading into SNPs derived from TMV and other rod-shaped viruses has been previously demonstrated^{11, 12, 14, 16}. However, in these systems, the association was achieved using chemical conjugation strategies, both before and after SNP formation. While these studies demonstrated functional conjugation of fluorophores and other classes of cargo, these covalent modification strategies rely on modification of the active ingredient. In the agricultural setting this is highly undesired, because changing the chemical structure of the active ingredient will require lengthy regulatory processes and increase costs in product development. Furthermore, many bioconjugation procedures are multi-step processes with intermediated and final purification steps. The processes can be time-consuming, reduce yields, and increase costs. A one-step synthesis of active-laden SNPs may mitigate these technological hurdles (outlined in FIG. 3A).

A fixed concentration of Cy5 (1 mg mL⁻¹) was added during the thermal transition (60 s, 96° C.) of TMGMV to SNP, ranging from 0.1- to 10-fold mass ratio compared to TMGMV. The resulting Cy5-SNPs were then purified over a desalting column to remove excess Cy5. The purified samples were visibly concentrated with Cy5 (FIG. 3B, inset). Cy5-SNPs were imaged by SEM and there were no apparent differences in the morphology of Cy5-SNPs compared to the non-loaded SNPs (FIG. 3B). The synthesis yielded fewer non-SNP aggregates, which may be attributed to the 5% by volume of DMSO added to the synthesis via Cy5, which may aid in the dissociation and thermal transformation of SNPs. This shows that this non-covalent encapsulation strategy does not interfere with the formation of SNPs. To quantify Cy5 encapsulation, Applicant analyzed the absorbance of the purified SNPs at 647 nm. Applicant found a TMGMV concentration-dependent removal of Cy5 (1 mg mL⁻¹) from the solution into the particle phase, with removal efficiencies ranging from 25% for 0.1 mg mL⁻¹ TMGMV to nearly 100% for 10 mg mL⁻¹ TMGMV (FIG. 3D). The encapsulation efficiencies were calculated are reported in FIG. 3F, showing high capacity at higher protein concentrations. While there is still room for optimization of loading efficiencies for each TMGMV concentration, Applicant demonstrated cargo-laden SNPs of distinct sizes from 100-200 nm to 500 nm-2 μm can be obtained in a one-step synthesis that does not require chemical alteration of the cargo. For Cy5, 77 molecules per CP were loaded in the 100-200 nm Cy5-SNPs (0.1 mg mL⁻¹ TMGMV). As TMGMV concentration and particle size increased, the amount of Cy5 per CP trended downward to fewer than 10 molecules per CP for the largest particles (500 nm-2 μm), still exceeding the maximum achieved through bioconjugation without and optimization.

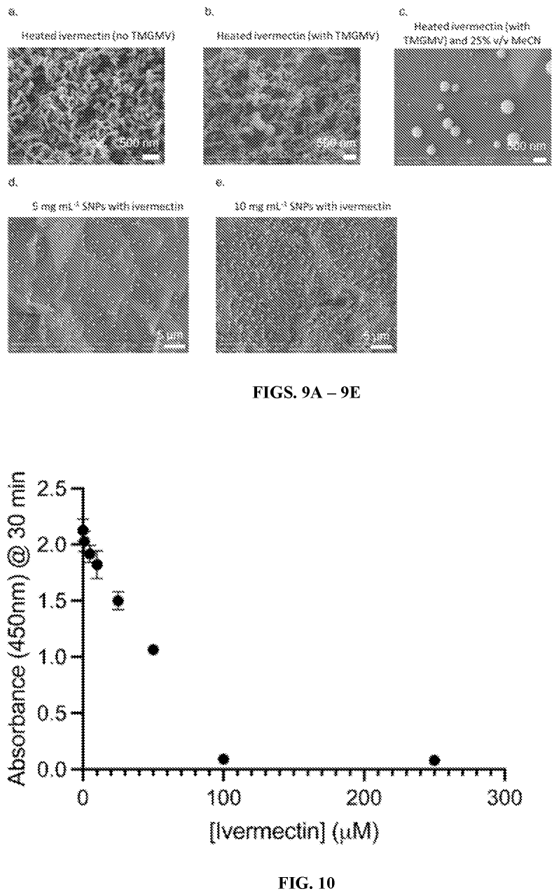

Next, ivermectin was encapsulated. Ivermectin has limited water solubility and was previously shown to be a poor choice for chemical conjugation²². Therefore Applicant tested encapsulation during the rod-to-SNP transition. When Applicant heated aqueous solutions containing ivermectin and TMGMV to entrap the active ingredient into SNPs, Applicant observed the formation of a fluffy colloidal byproduct which interfered with SNP formation (FIG. 9). This byproduct formed regardless of the presence of TMGMV, identifying it as an aggregate of ivermectin. To increase the solubility of ivermectin and enable SNP entrapment, Applicant added 25% by volume acetonitrile as a cosolvent. The use of the cosolvent indeed enabled hydrophobic cargo encapsulation and therefore greatly diversifies the types of cargo Applicant can entrap in SNPs. The cosolvent was then removed using sedimentation, dialysis, or other buffer exchange techniques. Applicant observed properly formed IVN-SNPs at all sizes (FIG. 3C and FIG. 9).

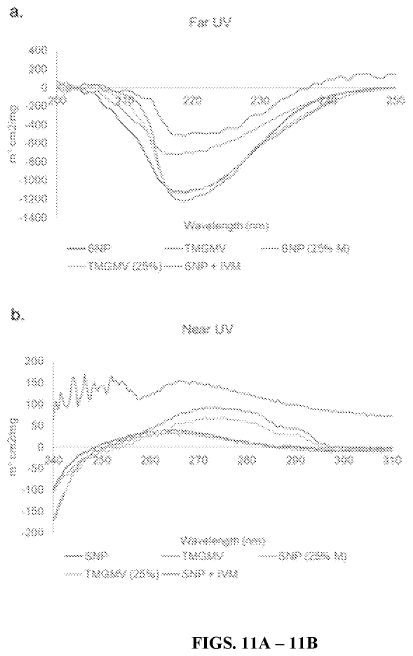

The amount of ivermectin per SNP formulation was determined using a competitive ELISA (FIG. 3E) and standard curve (FIG. 10). The ivermectin loading per CP was relatively higher when lower concentrations of TMGMV were used (FIG. 3F), matching the trends for Cy5. Up to ˜50 ivermectin molecules per CP was detectable for SNPs measuring 100-200 nm (0.1 mg mL⁻¹ TMGMV). For the smaller SNPs (100-200 nm), the estimated loading per SNP is on the order of 1×106 ivermectin per SNP, or 60% ivermectin by mass. To gain insights into the structural changes that occur during SNP formation with cosolvent, circular dichroism (CD) spectra comparing IVN-SNP vs. TMGMV were analyzed (FIG. 11).

When comparing TMGMV rods to SNPs, the data show a change in intensity of signal around 200-220 nm and a peak maximum shift from 270 nm to 260 nm after the thermal transition. Shifts associated with SNP formation are exacerbated in the presence of acetonitrile as a cosolvent, but acetonitrile without heating shows little effect on CD spectra. Additionally, the presence of ivermectin leaves a spectral fingerprint at 240-260 nm. Because TMV, a close homolog of TMGMV, undergoes large changes in secondary structure during SNP formation, these changes in TMGMV were expected as well.

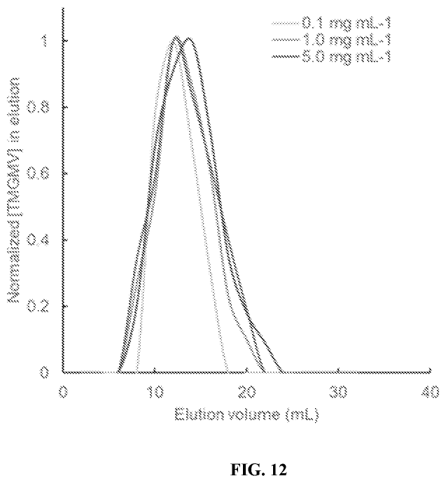

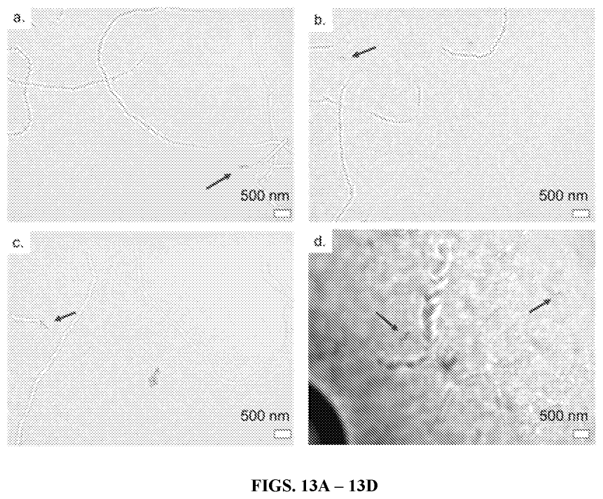

The effect of cargo loading on soil mobility was assessed next. Applicant confirmed soil mobility of the cargo-laden SNPs and the elution maxima matched with the empty SNPs, indicating that the cargo does not impact soil mobility (FIG. 3G, FIG. 12). Importantly, Applicant confirmed in 10 cm soil columns, the Cy5 signal in the eluent overlayed well with the protein signal for the Cy5-SNPs (this held true for all sizes tested, 500 nm-2 μm SNPs), indicating that the non-covalent loading into SNPs is robust and stable. This is in stark contrast for the TMGMV rods: here Applicant established non-covalent loading strategies by making use of electrostatic binding, while stable under physiological conditions, Applicant found the cargo to be stripped of the particle in soil⁷.

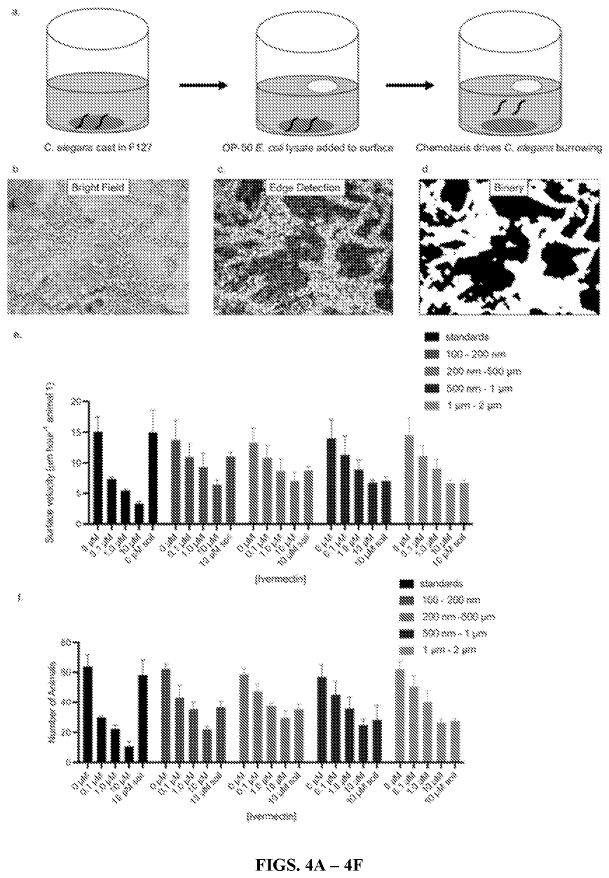

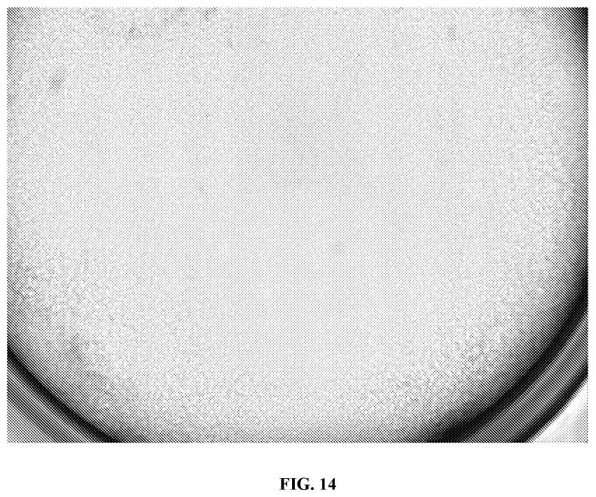

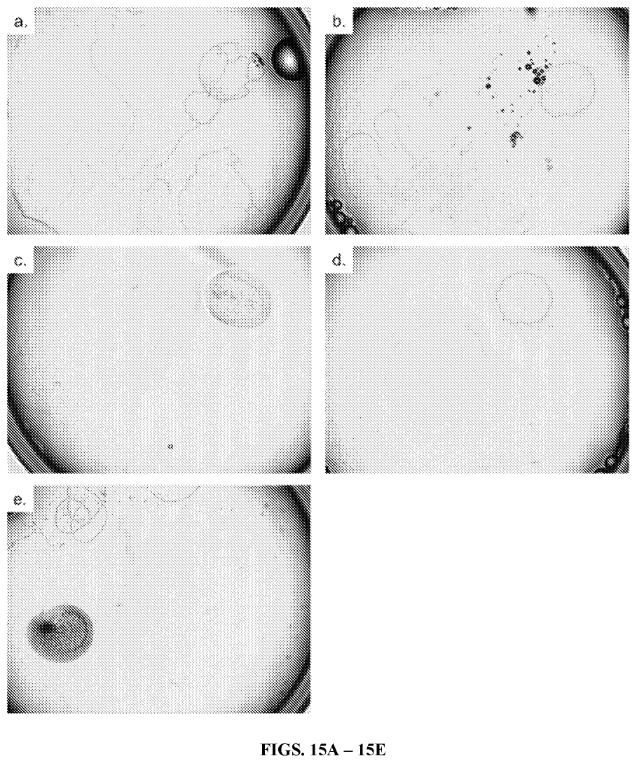

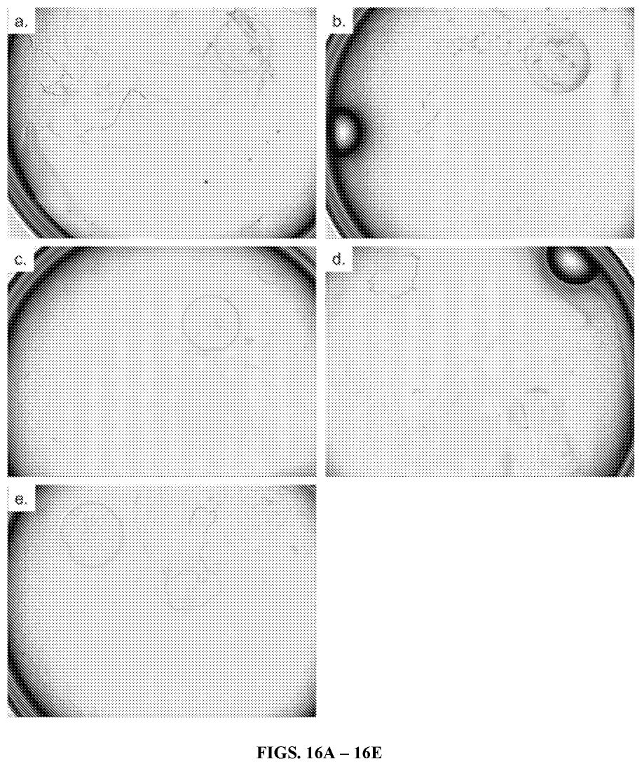

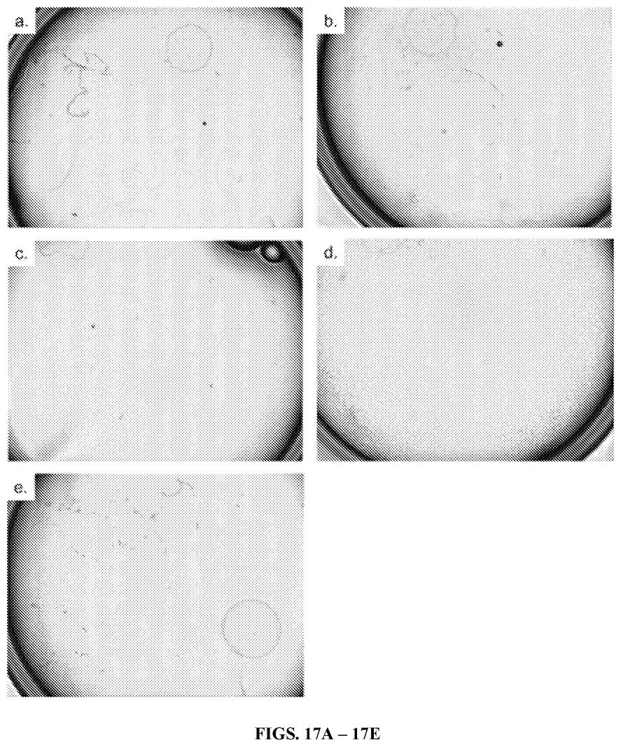

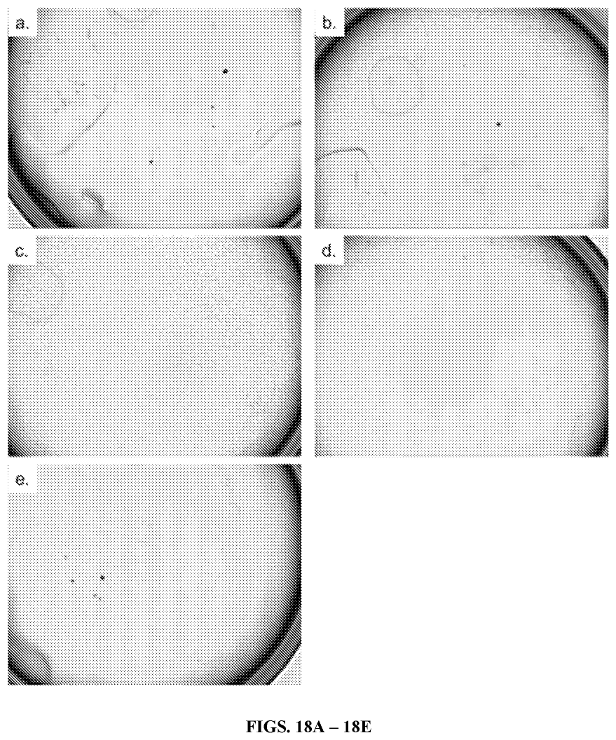

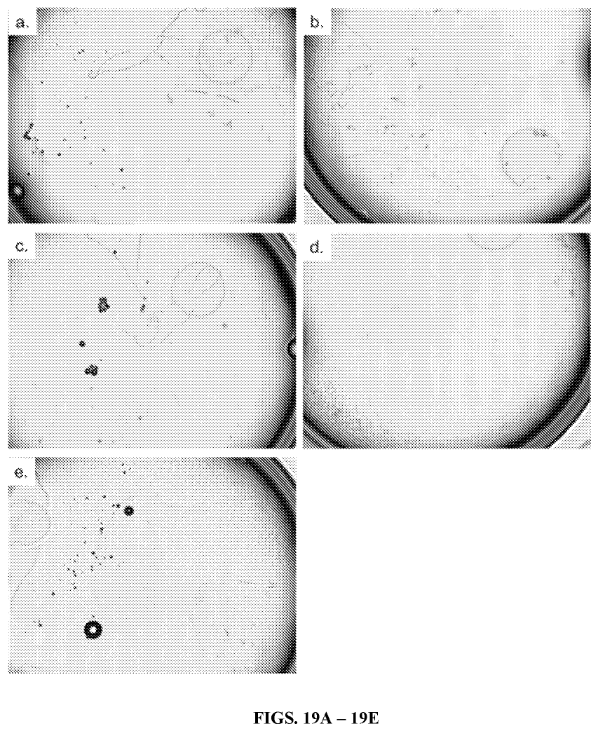

As a final test, Applicant delivered IVN-SNPs against C. elegans (FIG. 4). Nematodes were incubated with SNPs, IVN-SNPs, and free IVN for 90 minutes in solution. In parallel, the same (IVN-)SNPs were passed through a soil column and collected before treating the C. elegans in solution. For all samples, Applicant performed a modified gel burrowing assay (FIG. 4A) by transferring the nematodes and casting them in Pluronic F127 in a well-plate²³. The surface was coated with E. coli OP-50 lysate to attract the nematodes, and the number of C. elegans which penetrated to the surface were counted. Nematode mobility was quantified based on their total track coverage using edge detection (FIGS. 4B-4D, FIG. 13, FIG. 14). The mobility was then estimated using the following equation:

$$v = \frac{1}{N} \frac{A}{{A}_{0}} \frac{D}{Δ t}$$

where v is the approximated mobility, N is the total number of nematodes, A is the area of track coverage, A₀ is the total area in the image, D is the diameter of the well plate, and Δt is the time interval of the experiment (see FIG. 15-FIG. 19). Data demonstrated efficacy, as measured by reduction in burrowing capabilities of C. elegans, of soluble ivermectin and IVN-SNPs (FIG. 4E and FIG. 4F); SNPs alone had no effect which is as expected. Interestingly, the simple approximation of marked surface area as a proxy for number of nematodes on the surface correlated well, suggesting this method could be useful for future high throughput screens at a lower magnification. The highest tested concentration of ivermectin (10 μM) led to a 4.5-fold reduction in surface velocity and a 6-fold reduction in the number of nematodes on the surface.

Applicant also noted the largest SNPs (500-2 μm at 5 and 10 mg mL⁻¹) led to a >2-fold reduction in nematode surface velocity and a >2-fold reduction in number of nematodes on the surface. The smallest SNPs (100-200 nm at 0.1 mg mL⁻¹) yielded a 2.35-fold reduction in nematode surface velocity and 2.9-fold reduction in the number of nematodes on the surface. The ivermectin concentration in these experiments was normalized, therefore Applicant hypothesize that either the smaller SNPs interact more efficiently with the nematodes, either based on diffusion or higher nanoparticle-to-nematode ratio, and/or that ivermectin may be more accessible and adsorbed onto the surface of the SNPs (therefore possibly resulting in burst release).

The efficacy of IVN-SNP formulations was around 35% less effective than soluble ivermectin at 10 μM for the 500 nm-2 μm IVN-SNPs. However, in the agricultural setting, nematicides need to reach the root-feeding nematodes, therefore a realistic comparison needs to include the soil environment. Applicant added free ivermectin and IVN-SNPs onto a 10 cm soil column and collected fractions before treating C. elegans and conducting the burrowing assay. Only IVN-SNPs resulted in a significant reduction in the number of nematodes and their surface mobility, whereas soluble ivermectin showed no reduction (FIG. 4E, FIG. 4F). IVN-SNPs formed at 0.1 and 1.0 mg mL⁻¹ TMGMV resulted in a 1.36-fold and 1.72-fold reduction on surface velocity and a 1.74-fold and 1.81-fold reduction in nematode number. These data indicate a 2.25-fold (100-200 nm SNP) and 1.33-fold reduction (500 nm-1 μm) of efficacy after soil application at 10 μM ivermectin. This reduction in efficacy was not observed for the larger SNPs: IVN-SNPs at 5.0 and 10 mg mL⁻¹ showed similar efficacy before and after soil treatment, with a 2.14-fold reduction in surface velocity and 2.25-fold reduction in the number of nematodes for 5 mg mL⁻¹ after passing through a soil column and a 2.24-fold reduction in surface velocity and 2.30-fold reduction in nematode number for 10 mg mL⁻¹. From this data Applicant concluded that SNP entrapment of agrochemical cargo enabled soil mobility resulting in effective treatment of nematodes.

In summary, SNP-based encapsulation was highly efficient for soil-based delivery of nematicides. Applicant detailed the phase space for SNP synthesis, showing heating times greater than 30 s and temperatures higher than 90° C. led to SNP formation. SNP sizes were protein concentration dependent, and a size range of SNPs from 100 nm to 2 μm in diameter was synthesized. Applicant demonstrated effective encapsulation of Cy5 and ivermectin into SNPs during thermal TMGMV-to-SNP transition. Finding a medium, like acetonitrile, which can be heated to >90° C. and maintain target molecule solubility is important for effective encapsulation. Future applications may require more sustainable cosolvents. Acetonitrile enhanced solubility of the active ingredient and improved SNP synthesis, likely by aiding destabilization of the TMGMV assembly. Importantly, Applicant demonstrated efficient active ingredient loading and co-delivery in soil to target root-feeding nematodes. While free ivermectin was not effective after soil passage, IVN-SNPs facilitated soil mobility and efficacy against C. elegans. Applicant envision these techniques could find applications beyond pesticide delivery in soil and be extended for foliar active ingredient delivery. TMGMV itself has already been approved by the EPA as a bioherbicide and this non-covalent encapsulation could to streamline the regulatory process. The use of one-pot synthesis methods is expected to yield in a scalable industry process.

### EXPERIMENTAL

### TMGMV Preparation

TMGMV from BioProdex (Gainesville, FL, USA) was prepared as previously described and stored at −20° C. until use (Chariou, P. L. et al., Nature Nanotechnology, 2019; Chariou, P. L. et al., ACS Agricultural Science & Technology, 2021; Chariou, P. L. et al., ChemBioChem, 2022; González-Gamboa, I. et al., ChemBioChem, 2022). In brief, thawed TMGMV was dialyzed against potassium phosphate buffer (KP; 10 mM, pH 7.2) for 24 hours at 4° C. using 12-14 kDa dialysis tubing (Fisher Scientific S432700, Waltham, MA, USA), and the process was repeated with fresh buffer the following 48 hours. The virus solution was centrifuged at 10,000×g for 20 min (Beckman Coulter Allegra centrifuges, Brea, CA, USA). The supernatant was collected and ultracentrifuged at 42,000 rpm for 2.5 hours at 4° C. (Beckman Coulter Optima L-90k Ultracentrifuge with 50.2 Ti rotor, Brea, CA, USA). The pellet was resuspended under overnight at 4° C. in KP buffer. The concentration was determined using a Nanodrop 2000 (Thermo Scientific, Waltham, MA, USA).

### SNP Preparation

TMGMV solutions were diluted to the desired concentration (0.1, 0.3, 0.5, 1.0, 5.0, or 10.0 mg mL-1) in DI H₂O in PCR tubes at a volume of 100 μL. A thermocycler (MJ Research PTC200, Waltham, MA, USA) was used to program the run to heat to the desired temperature (60, 70, 80, 90, 96, and 98° C.) for a fixed duration (5, 10, 15, 20, 30, 60, and 120 s) before being lowered to 10° C. Samples were stored at 4° C. between measurements.

### SEM

Particle preparations were dried on a silica wafer bound to a stainless-steel mounting stud by carbon tape. The sample was left to dry overnight before iridium coating with 12 seconds of coating time. The samples were imaged in a Zeiss 500 at 3-5 kV under vacuum.

### SDS-PAGE

Denatured TMGMV SNP samples (10 μg) were loaded on a 12% NuPAGE gel (Life Technologies, Carlsbad, CA, USA) and run on 1×MOPS Running Buffer (Life Technologies, Carlsbad, CA, USA). Cyanine 5 encapsulated in SNPs was visualized under UV light. Gel Code Blue stain (Life Technologies, Carlsbad, CA, USA) was used to stain coat proteins and they were visualized under white light in a gel imager (ProteinSimple, Santa Clara, CA, USA).

### Soil Mobility Assays

Magic Topsoil (Michigan Peat) was weighed and packed into a cylindrical column fitted with miracloth at the bottom. The bottom of the tube had a tapered outlet for dropwise fraction collection. The soil was wetted with DI H2O and left to stop dripping. An aliquot of 0.5 mg TMGMV or SNPs was added to the column and irrigated at a constant rate of 5 mL min-1 using a syringe pump with DI H2O. The effluent was collected in 2 mL fractions and run on SDS PAGE for analysis. The band intensity of the fractions on SDS PAGE was determined using ImageJ.

### Encapsulation of Small Molecules in SNPs

A solution of small molecule was prepared at 5 mg mL-1 (Cy5 in DI H2O and ivermectin in acetonitrile). These solutions were diluted to 1 mg mL-1 small molecule (20% acetonitrile v/v for ivermectin) in the desired concentration of TMGMV in DI H2O (0.1, 0.3, 1.0, 5.0, 10 mg mL-1 final TMGMV concentration). The samples were then prepared on a thermocycler as previously described. Free/non-encapsulated small molecules were removed by spin filtration (EMD Millipore UFC500324 3 kDa, Burlington, MA, USA) at 5000×g for 5 minutes, the process was repeated twice. The retentate was diluted in DI H2O. The sample absorbance of Cy5 samples was collected using a nanodrop. The concentration of Cy5 in the samples was determined using the Beer Lambert Law with the absorbance at 647 nm and the molar extinction coefficient of 230,400 cm-1 M-1.

### Competitive ELISA for Ivermectin Quantification

Samples containing ivermectin were diluted according to the manufacturer's protocols using a High-Sensitivity Ivermectin ELISA kit (Creative Diagnostics DEIASL215, Shirley, NY, USA). A competitive ELISA for ivermectin was conducted in the provided 96-well plate. Ivermectin-Horseradish peroxidase conjugates in the sample wells then reacted with the provided substrate for 30 minutes before quenching with equal volume of 1N HCl. Absorbance was measured on a plate reader (Tecan Infinite M Plex, San Jose, CA, USA) at 450 nm and concentrations were fitted to a standard curve of ivermectin.

### Circular Dichroism

An Aviv model 215 CD spectrometer was used to collect CD spectra. A quartz cuvette with a path length of 2 mm (Starna Cells, Atascadero, CA, USA) at 25° C. was used for each sample and each measurement was collected twice. Sample concentrations between 0.025 mg/mL to 0.5 mg/mL were used to obtain a volume of 400 μL for each CD run. Two separate scans collected the near and far UV spectra. 250 nm to 180 nm with a wavelength step of 1 nm and an averaging time of 1 second was used for far UV spectra. For near UV spectra, 310 nm to 240 nm was scanned with a wavelength step size of 0.5 nm and an averaging time of 1 second.

C. elegans Cultivation

The N2 strain of Caenorhabditis elegans was obtained from Caenorhabditis Genetics Center (University of Minnesota, Minneapolis, MN, USA) grown on solid Nematode Growth Medium (NGM) agar plates with OP-50 E. coli as the primary food source. The animals were grown and maintained at 20° C.

C. elegans Treatment with Ivermectin and IVN-SNPs

A concentrated solution of C. elegans was aliquoted with ˜100 animals per microcentrifuge tube. The animals were centrifuged briefly for 30 s at 200×g to create a pellet, the supernatant was removed and replaced with a solution specific to the treatment group, such as with SNPs or ivermectin. The C. elegans were incubated for 90 minutes in these solutions before being transferred to the burrowing assay (Lesanpezeshki, L. et al., Scientific Reports 2019).

### Pluronic-Based Burrowing Assays

Using the optimized Pluronic concentration of 26% w/w previously reported, Pluronic F127 solutions were stored at 4° C. to dissolve and stay soluble (Lesanpezeshki, L. et al., Scientific Reports 2019). The Pluronic F127 solutions were kept at on ice while preparing the assay. 30 μL Pluronic F127 was added to a 6-well plate. A concentrated solution of treated C. elegans was deposited to introduce around 100 animals. After around 10 minutes, a layer of 2 mL Pluronic was cast on top. A solution of OP-50 E. coli lysate (20 μL of OD600=0.5) was added to the surface. After 2 hours, an 11×11 area scan at 4× magnification (Keyence BZ-X800 Fluorescence Microscope) was taken for each well. The number of nematodes on the surface were counted manually and the area fraction of their tracks was determined by edge detection and binary image transformation in ImageJ.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Agriculture, U. N. F. a. Hunger and food insecurity; United Nations: https://www.fao.org/hunger/en/, 2023.
2. Arias-Estévez, M.; López-Periago, E.; Martínez-Carballo, E.; Simal-Gándara, J.; Mejuto, J.-C.; García-Río, L., The mobility and degradation of pesticides in soils and the pollution of groundwater resources. Agriculture, Ecosystems & Environment 2008, 123 (4), 247-260.
3. Brewer, A.; Dror, I.; Berkowitz, B., The Mobility of Plastic Nanoparticles in Aqueous and Soil Environments: A Critical Review. ACS ES&T Water 2021, 1 (1), 48-57.
4. Chariou, P. L.; Dogan, A. B.; Welsh, A. G.; Saidel, G. M.; Baskaran, H.; Steinmetz, N. F., Soil mobility of synthetic and virus-based model nanopesticides. Nature Nanotechnology 2019, 14 (7), 712-718.
5. Katagi, T., Soil Column Leaching of Pesticides. In Reviews of Environmental Contamination and Toxicology Volume 221, Whitacre, D. M., Ed. Springer New York: New York, NY, 2013; pp 1-105.
6. Beckers, S. J.; Staal, A. H. J.; Rosenauer, C.; Srinivas, M.; Landfester, K.; Wurm, F. R., Targeted Drug Delivery for Sustainable Crop Protection: Transport and Stability of Polymeric Nanocarriers in Plants. Advanced Science 2021, 8 (11), 2100067.
7. Chariou, P. L.; Steinmetz, N. F., Delivery of Pesticides to Plant Parasitic Nematodes Using Tobacco Mild Green Mosaic Virus as a Nanocarrier. ACS Nano 2017, 11 (5), 4719-4730.
8. Charudattan, R.; Hiebert, E., A Plant Virus as a Bioherbicide for Tropical Soda Apple, Solanum viarum. Outlooks on Pest Management 2007, 18 (4), 161-171.
9. Chariou, P. L.; Ma, Y.; Hensley, M.; Rosskopf, E. N.; Hong, J. C.; Charudattan, R.; Steinmetz, N. F., Inactivated Plant Viruses as an Agrochemical Delivery Platform. ACS Agricultural Science & Technology 2021, 1 (3), 124-130.
10. Shin, M. D.; Hochberg, J. D.; Pokorski, J. K.; Steinmetz, N. F., Bioconjugation of Active Ingredients to Plant Viral Nanoparticles Is Enhanced by Preincubation with a Pluronic F127 Polymer Scaffold. ACS Applied Materials & Interfaces 2021, 13 (50), 59618-59632.
11. Nikitin, N.; Ksenofontov, A.; Trifonova, E.; Arkhipenko, M.; Petrova, E.; Kondakova, O.; Kirpichnikov, M.; Atabekov, J.; Dobrov, E.; Karpova, O., Thermal conversion of filamentous potato virus X into spherical particles with different properties from virions. FEBS Letters 2016, 590 (10), 1543-1551.
12. Manukhova, T. I.; Evtushenko, E. A.; Ksenofontov, A. L.; Arutyunyan, A. M.; Kovalenko, A. O.; Nikitin, N. A.; Karpova, O. V., Thermal remodelling of Alternanthera mosaic virus virions and virus-like particles into protein spherical particles. PLOS ONE 2021, 16 (7), e0255378.
13. Atabekov, J.; Nikitin, N.; Arkhipenko, M.; Chirkov, S.; Karpova, O., Thermal transition of native tobacco mosaic virus and RNA-free viral proteins into spherical nanoparticles. J Gen Virol 2011, 92 (Pt 2), 453-6.
14. Bruckman, M. A.; Czapar, A. E.; VanMeter, A.; Randolph, L. N.; Steinmetz, N. F., Tobacco mosaic virus-based protein nanoparticles and nanorods for chemotherapy delivery targeting breast cancer. Journal of Controlled Release 2016, 231, 103-113.
15. Bruckman, M. A.; Hern, S.; Jiang, K.; Flask, C. A.; Yu, X.; Steinmetz, N. F., Tobacco mosaic virus rods and spheres as supramolecular high-relaxivity MRI contrast agents. Journal of Materials Chemistry B 2013, 1 (10), 1482-1490.
16. Bruckman, M. A.; VanMeter, A.; Steinmetz, N. F., Nanomanufacturing of Tobacco Mosaic Virus-Based Spherical Biomaterials Using a Continuous Flow Method. ACS Biomaterials Science & Engineering 2015, 1 (1), 13-18.
17. Deutsch, D. G., Effect of prolonged 100° C. heat treatment in sodium dodecyl sulfate upon peptide bond cleavage. Analytical Biochemistry 1976, 71 (1), 300-303.
18. Kowit, J. D.; Maloney, J., Protein cleavage by boiling in sodium dodecyl sulfate prior to electrophoresis. Analytical Biochemistry 1982, 123 (1), 86-93.
19. Kurien, B. T.; Scofield, R. H., Common Artifacts and Mistakes Made in Electrophoresis. In Protein Electrophoresis: Methods and Protocols, Kurien, B. T.; Scofield, R. H., Eds. Humana Press: Totowa, NJ, 2012; pp 633-640.
20 Dobrov, E. N.; Nikitin, N. A.; Trifonova, E. A.; Parshina, E. Y.; Makarov, V. V.; Maksimov, G. V.; Karpova, O. V.; Atabekov, J. G., B-structure of the coat protein subunits in spherical particles generated by tobacco mosaic virus thermal denaturation. Journal of Biomolecular Structure and Dynamics 2014, 32 (5), 701-708.
21. Della Pelle, G.; Delgado López, A.; Salord Fiol, M.; Kostevšek, N., Cyanine Dyes for Photo-Thermal Therapy: A Comparison of Synthetic Liposomes and Natural Erythrocyte-Based Carriers. International Journal of Molecular Sciences 2021, 22 (13), 6914.
22. Ahmadpour, E.; Godrati-Azar, Z.; Spotin, A.; Norouzi, R.; Hamishehkar, H.; Nami, S.; Heydarian, P.; Rajabi, S.; Mohammadi, M.; Perez-Cordon, G., Nanostructured lipid carriers of ivermectin as a novel drug delivery system in hydatidosis. Parasites & Vectors 2019, 12 (1), 469.
23. Lesanpezeshki, L.; Hewitt, J. E.; Laranjeiro, R.; Antebi, A.; Driscoll, M.; Szewczyk, N. J.; Blawzdziewicz, J.; Lacerda, C. M. R.; Vanapalli, S. A., Pluronic gel-based burrowing assay for rapid assessment of neuromuscular health in C. elegans. Scientific Reports 2019, 9 (1), 15246.

1. A virus-like particle (VLP) derived from Tobacco mild green mosaic virus (TMGMV) or a derivative thereof optionally conjugated to an agent.

2. The VLP of claim 1, wherein the agent or derivative thereof is entrapped in the VLP or covalently attached to the VLP.

3. The VLP of claim 1, wherein the diameter of the VLP is from about 100 nm to about 2 μm.

4. The VLP of claim 2, wherein the diameter of the VLP is from about 100 nm to about 200 nm.

5. The VLP of any of claim 1, wherein the agent is loaded at a concentration of about 5 mg mL-1 to about 10 mg mL-1 or from about 0.5 mg mL-1 to about 0.2 mg mL-1.

6. (canceled)

7. The VLP of claim 3, wherein the agent is loaded at a concentration of from about 5 mg mL⁻¹ to about 10 mg mL⁻¹.

8. The VLP of claim 3, wherein the agent is loaded at a concentration of from about 0.5 mg mL-1 to about 0.2 mg mL-1.

9. The VLP of claim 4, wherein the agent is loaded at a concentration of from about 5 mg mL-1 to about 10 mg mL-1 or from about 0.5 mg mL-1 to about 0.2 mg mL-1.

10. (canceled)

11. The VLP of claim 1, wherein the agent is an insoluble compound or a pesticide.

12. The VLP of claim 11, wherein the pesticide is an avermectin.

13. The VLP of claim 12, wherein the avermectin is selected from the group of ivermectin, abamectin, doramectin, eprinomectin, moxidectin, or selamectin.

14. The VLP of claim 1, wherein the agent is ivermectin.

15. A plurality of VLPs of claim 1, wherein the VLPs are the same or different from each other.

16. The plurality of VLPs of claim 15, wherein the agents are the same or different from each other.

17. The plurality of VLPs of claim 1, wherein the TMGMV are the same or different from each other.

18. A composition comprising the VLP claim 1, and a carrier, optionally a carrier for agricultural application or delivery.

19. The composition of claim 18, further comprising a preservative or stabilizer and optionally wherein the composition is lyophilized or frozen.

20. (canceled)

21. A method for treating an agricultural environment, comprising administering to environment the VLP of claim 1.

22. The method of claim 21, wherein the administration is to soil, feed, or a plant leaf, stem, flower or root.

23. A kit comprising the VLP of claim 1, and instructions for use.
